(19) United States
(12) Patent Application Publication
(10) Pub. No.: US 2025/0019262 A1
(43) Pub. Date: Jan. 16, 2025

(54) Method and Apparatus for TOC Monitoring of Purified Water

(71) Applicant: VWS (UK) Limited, High Wycombe Buckinghamshire (GB)

(72) Inventors: John Walker, Caversham Berkshire (GB); Craig Stephen Over, High Wycombe Buckinghamshire (GB); Lee Underwood, High Wycombe Buckinghamshire (GB); Peter Bower, High Wycombe Buckinghamshire (GB)

(21) Appl. No.: 18/688,122

(22) PCT Filed: Jul. 28, 2022
(86) PCT No.: PCT/GB2022/051980

§ 371 Date: Feb. 29, 2024

(30) Foreign Application Priority Data
Sep. 1, 2021 (GB) 2112446.6

(51) Int. Cl. C02F 1/00; C02F 1/32; C02F 1/42; C02F 1/469; C02F 1/68; C02F 9/00; G01N 27/08; G01N 33/18

(52) U.S. Cl. CPC C02F 1/008; C02F 9/00; G01N 27/08; G01N 33/1826; C02F 1/32; C02F 1/42; C02F 1/4691; C02F 1/68; C02F 2209/05; C02F 2209/20; C02F 2301/043; C02F 2301/046; C02F 2301/08

(57) ABSTRACT

The present invention relates to a method of determining the total organic carbon content (TOC) of a purified water stream in a water purification apparatus having at least a first pump, a first conductivity sensor, an oxidiser, and an oxidiser recirculation circuit having a dedicated second pump, the method comprising at least the steps of:
(a) using the first pump to pass a water supply stream through the water purification apparatus, including through the first conductivity sensor and the oxidiser, to provide a purified water stream available for dispense;
(b) using the first conductivity sensor to measure a first conductivity value of the water supply stream prior to the oxidiser;
(c) stopping the first pump;
(d) using the second pump in the oxidiser recirculation circuit to recirculate the water in the oxidiser only through the first conductivity sensor and the oxidiser a plurality of times,
(e) using the first conductivity sensor to measure a second conductivity value of the recirculated water; and
(f) calculating the TOC of the water in the oxidiser prior to step (d), from the first and second conductivity values.

The present invention relates to a method for monitoring the total organic carbon content (hereinafter “TOC”) of water in and being dispensed from water purification apparatus and units, particularly but not exclusively for laboratory water, and for the verification and calibration of such apparatus.

### BACKGROUND

Water purification apparatus and units for use in laboratories and healthcare facilities are well known. Generally, they involve the reduction and/or removal of contaminants and impurities to very low levels. They typically contain a variety of technologies that remove particles, colloids, bacteria, ionic species and organic substances and/or molecules.

The levels of such contaminants and impurities can be monitored in various ways, one being to measure the total organic carbon content (“TOC”) as a measure of any remaining organic substances in the water. TOC limits for various purities or grades of water are prescribed by various national and international bodies, for example the US and European pharmacopoeias (USP/EP), the American Society for Testing and Materials (ASTM) and the Clinical Laboratory Standards Institute (CLSI).

Water contaminants and impurities that are measured to define the TOC are typically broken down by the use of an oxidiser, to oxidise such contaminants and impurities, typically forming carbon dioxide (CO2) from the carbon content. The increase in conductivity of the water is measured, and an algorithm applied to determine the amount of TOC that was present. The carbon dioxide can then be removed by subsequent deionisation.

Dedicated TOC monitors are well known in the art; see for example the monitor shown in WO99/42824A1.

WO2010/043896A discloses a method of determining the TOC of a purified water stream so that the TOC of the purified water stream can be provided without requiring a dedicated TOC monitor at the point of dispense of the purified water stream.

However, TOC oxidisers have a fixed maximum quantity of potential to oxidise, and dedicated TOC monitors rely on the oxidiser having the capacity to oxidise all of the organic molecules to carbon dioxide.

In conventional laboratory water purification apparatus, a UV lamp, typically used as an oxidiser, is relatively low powered as it usually only has to oxidise low levels of dissolved organic material in order for the equipment to function properly. Hence oxidisers with the ability to oxidise and accurately determine TOC of <50 ppbC are typically incorporated into such apparatus.

However, where the level of TOC is beyond the capability of the oxidiser, further increases in TOC do not result in the measurement of any increase in oxidation across the oxidiser. This means that there is a maximum range of the TOC monitor using a particular oxidiser at particular flow conditions.

Pharmacopoeia system suitability tests such as USP643 and EP 2.2.44 now specify that apparatus for measuring TOC must be able to “periodically demonstrate” that they can provide a suitable response when fed with a level of 500 ppbC of specified organic molecules (as defined therein).

It is known in the art that in a short wavelength UV (such as 185 nm from a mercury lamp, or 172 nm from a Xenon Excimer sources) has a very low penetration depth into a purified water stream (measured in the low nanometers), and so the bulk of the oxidation within an oxidiser using UV at these or other similar wavelengths, takes place only in a very shallow area of an oxidiser, close to the light source. It is impractical and too expensive to make oxidisers with a water path so that all of the water is exposed to oxidation area (due to the precision of manufacture and pressure required to pass the water stream through the oxidiser).

Furthermore, over time, oxidisers may lose efficiency as their component parts age. Thus, the maximum oxidation possible may become lowered, and so the corresponding maximum conductivity difference will also become lower. Measurement of a TOC level of an ‘old’ maxima would instead give a result of a new maxima. In a system where only a percentage of the TOC is oxidised by the oxidiser and a calibration is applied to the conductivity change, a reduction in oxidation efficiency may also reduce the percentage of oxidation that will take place for a particular set of conditions. The conductivity difference detected by the line-cells may therefore be reduced, and require a change in the algorithm for the correct TOC result to be given.

As a result, oxidisers within water purification apparatus should be replaced well in advance of complete exhaustion of their useful life, to avoid lower than expected oxidation occurring, and false TOC readings being given by the monitoring process.

It is an object of the present invention to provide an improved method of monitoring the TOC of water being purified in a water purification apparatus or unit, to help monitor the performance of the oxidiser and to help accommodate measurement of higher TOC levels.

### SUMMARY OF THE INVENTION

Thus, according to one aspect of the present invention, there is provided a method of determining the total organic carbon content (TOC) of a purified water stream in a water purification apparatus having at least a first pump, a first conductivity sensor, an oxidiser, and an oxidiser recirculation circuit having a dedicated second pump, the method comprising at least the steps of:
(a) using the first pump to pass a water supply stream through the water purification apparatus, including through the first conductivity sensor and the oxidiser, to provide a purified water stream available for dispense;
(b) using the first conductivity sensor to measure a first conductivity value of the water supply stream prior to the oxidiser;
(c) stopping the first pump;
(d) using the second pump in the oxidiser recirculation circuit to recirculate the water in the oxidiser only through the first conductivity sensor and the oxidiser a plurality of times,
(e) using the first conductivity sensor to measure a second conductivity value of the recirculated water; and
(f) calculating the TOC of the water in the oxidiser prior to step (d), from the first and second conductivity values.

According to another aspect of the invention, there is provided a water purification apparatus comprising:
a first pump,
a first conductivity sensor,
an oxidiser, and
an oxidiser recirculation circuit having a dedicated a second pump,
   wherein the first pump is able to pass a water supply stream through the water purification apparatus, including through the first conductivity sensor and the oxidiser, to provide a purified water stream available for dispense;
the first conductivity sensor is able to measure a first conductivity value of the water supply stream prior to the oxidiser;
the second pump is only able to recirculate water through the oxidiser recirculation circuit, the first conductivity sensor and the oxidiser a plurality of times, to allow the first conductivity sensor to measure a second conductivity value of the recirculated water prior to the oxidiser; and
the first conductivity value is able to be compared with the second conductivity value to calculate a TOC of the water in the oxidiser prior to operation of the second pump.

According to another aspect of the present invention, there is provided a water purification system comprising an initial water purification apparatus able to provide a water supply stream having a conductivity <30 μS/cm, and a water purification apparatus as defined herein.

### BRIEF DESCRIPTION OF THE DRAWINGS

FIG. 1 is a schematic diagram of a method and apparatus for determining the total organic carbon content (TOC) of a purified water stream in a water purification apparatus according to one embodiment of the present invention; and

FIG. 2 is a graph of the conductivity over time as measured by a conductivity sensor in an oxidiser re-circulation circuit.

### DETAILED DESCRIPTION OF THE INVENTION

The present invention relates to a method of determining the total organic carbon content (TOC) of a purified water stream in a water purification apparatus having at least a first pump, a first conductivity sensor, an oxidiser, and an oxidiser recirculation circuit having a dedicated second pump, the method comprising at least the steps of:
(a) using the first pump to pass a water supply stream through the water purification apparatus, including through the first conductivity sensor and the oxidiser, to provide a purified water stream available for dispense;
(b) using the first conductivity sensor to measure a first conductivity value of the water supply stream prior to the oxidiser;
(c) stopping the first pump;
(d) using the second pump in the oxidiser recirculation circuit to recirculate the water in the oxidiser only through the first conductivity sensor and the oxidiser a plurality of times,
(e) using the first conductivity sensor to measure a second conductivity value of the recirculated water; and
(f) calculating the TOC of the water in the oxidiser, prior to step (d), from the first and second conductivity values.

When the quantity of the TOC in the water feed supply is desired to be known, then the method further comprises providing the water in the oxidiser recirculation circuit for step (d) from the water supply stream that entered the water purification apparatus in step (a).

When the quantity of the TOC in the purified water stream available for dispense at an outlet is desired to be known, then the method further comprises providing the water in the oxidiser recirculation circuit for step (d) from the purified water stream available for dispense.

In an another embodiment of the present invention, water to be tested from another source could be added into a suitable cartridge, so that the method further comprises the step of inserting such a water-filled cartridge into the water purification apparatus upstream of the first conductivity sensor prior to step (d), and using said water in step (d). In this way, the present invention can be used to test the conductivity and TOC of a separate water source, such as a different water purification apparatus, or different water source or sample that only requires such analysis.

When a ‘system suitability test’ as per USP643 (and described in more detail hereinafter) is required to be operated, the water of the required content can be passed into the oxidiser recirculation circuit prior to (c) without treatment.

The present invention allows replacement of a failing oxidiser in the water purification apparatus to be correctly judged.

The present invention also allows the oxidiser to be calibrated to read high level TOCs, such as up to 500 ppbC.

The present invention also allows for calibration of the TOC measurement to maintain accuracy of results provided.

The water supply stream may comprise any source of water, generally being a pre-purified water source of conductivity <30 μS/cm, such as from a mains supply that has been pre-treated by a deionisation process such as reverse osmosis, capacitive deionisation or ion exchange. Commonly, such a water source is provided from a water pre-purification device which is in turn connected to a tap or other standard supply device, and includes a line or other connection from the water pre-purification device to with the water purification apparatus, (or more correctly “water ultra-purification and dispensing apparatus”).

Thus, the present invention can be serial to or incorporated with or additional to an initial or first water purification method or apparatus able to pre-treat a water stream. The present invention can ‘polish’ such pre-treated water. Such pre-treated water could be supplied from a reservoir.

Preferably, the present invention is integral with an initial or first water purification method or apparatus able to provide a water supply stream having a conductivity <30 μS/cm.

Thus, the present invention also provides a water purification system comprising an initial water purification apparatus, as known in the art and as discussed hereinabove, and able to provide a water supply stream having a conductivity <30 μS/cm, and a water purification apparatus as defined herein.

The water purification apparatus of the present invention may comprise any number of devices, parts, components, lines, etc., including but not limited to one or more of the following: pumps, meters, sensors, oxidisers, de-ionisers, valves, drains, control units and mechanisms, taps, filters, membranes.

The oxidiser in step (a) oxidises the water passing therethrough as part of the purification of the water supply stream.

One common oxidiser involves the use of ultraviolet light, and the short wavelength ultraviolet treatment of water for decomposing organic compounds or substances in water is well known in the art. Generally, ultraviolet light is able to decompose many organic compounds and substances that are contained or are residues in generally available water, by oxidising them to form ionisable or charged species, such as carbon dioxide, and water. Apparatus and instruments for providing suitable ultraviolet light are well known in the art, and typically involve emitting ultraviolet light at one or more specific wavelengths, such as at 185 nanometres, in an area or space through which the water passes.

In many water purification units or apparatus, oxidisers are provided as a distinct component, typically a separable component such as a replaceable lamp or module, having an ultraviolet emitter therein close to which the water stream passes from an inlet to an outlet. The purification of water in the present invention may involve one or more oxidisers, being in series, parallel or both.

Ionic or ionisable species created by the or each oxidiser are generally removed from the water stream, to provide a purified water stream, by the use of one or more de-ionisers. Many types and forms of de-ionisers are known in the art, and include, but are not limited to, one or more of the following; (electro) deionisation apparatus or units, or ion-exchanger resins. The action and operation of a de-ioniser is well known in the art, and they are not further described in detail herein.

A water purification apparatus or unit may comprise a plurality of de-ionisers, including one or more “pre-treatment” ion exchangers upstream of an oxidiser, as well as one or more ion-exchangers downstream of the oxidiser.

In general, a water purification apparatus or unit of the present invention only provide up to 1000 litres of purified water per hour, such as up to 51/min.

Such water purification apparatus are generally ‘stand alone’ units, generally only requiring connection to nearby water, preferably pre-treated water, and electricity supplies to be operable. Thus, they are generally independent and/or movable units operating in or at a specific location such as a laboratory. Preferably, at least the majority of the purification actions or processes occur within a housing or chassis. They are intended to provide a purified water stream only, such stream not being in combination with any other substance or compound, in the vicinity of where it is to be used.

The purified water stream available for dispense provided by step (a) is created by the reduction and/or removal of any or one or more of the contaminants and impurities in the supply water stream. This can involve the reduction and/or removal of one or more of the following: particles, colloids, bacteria, micro-organisms, ionic species, organic substances, dissolved gases.

In general, water purification apparatus and units are intended to provide a purified water stream having a conductivity of less than 1 μS/cm, preferably less than 0.1 μS/cm, and more preferably less than 0.067 μS/cm. This can be equated to a purified water stream having a resistivity of at least 1 MΩ-cm, preferably at least 10 MΩ-cm, more preferably at least 15 MΩ-cm. Additionally, purity specifications can be made for organic species to content levels of less than 500 ppb of total organic carbon (TOC), preferably less than 50 ppb; bacteria to levels less than 100 colony forming units (cfu) per millilitre, preferably less than 1 cfu/ml; and for dissolved oxygen and/or particles.

The skilled man is aware of the relationship between conductivity and resistivity, such that either one or both measurements can be made by a suitable measurer or meter. Thus, the term “conductivity value” as used herein relates to the measurement of the conductivity and/or resistivity of a water stream, either one or both of which may be used to provide a determination of TOC.

The skilled man is also aware that conductivity and/or resistivity measurements or values are temperature dependent. Commonly, a temperature of 25° C. is used as a standard temperature when discussing and comparing conductivity and/or resistivity measurements, such that the conductivity of “pure” water is considered to be 0.055 μS/cm and the resistivity is considered to be 18.2 MΩ-cm, at 25° C.

The dispense of at least a portion of the purified water stream can be provided through any form or type of outlet or outlets, optionally being co-ordinated or separate.

The water purification apparatus may have a dispense mode or other such form of operation, and a recirculation mode. Preferably, the point of dispense involves at least one valve, more preferably operable between a dispense position and a recirculating position. One or more valves may also provide control over the volume and/or rate of flow of the purified water stream at the point of dispense.

The dispense may involve the dispense of all of the purified water stream being provided by the water purification apparatus, such as whilst the water purification apparatus is in a dispense mode. Optionally, a portion of the purified water stream may be contemporaneously or simultaneously recirculated through at least a portion of the water purification apparatus whilst the remainder of the purified water stream is being dispensed.

The movement of water streams through a water purification apparatus is generally provided by the use of one of more pumps known in the art, and not further discussed in detail herein. Such pumps are water pumps.

Stopping of the dispense of the purified water stream may be carried out by the operation (usually through a controller) of one or more parts or components of the water purification apparatus, generally operation of one or more valves at or near the dispense, such as a 2-way valve able to move between a dispense position and a recirculating position.

Recirculating a purified water stream through at least a portion of a water purification apparatus is well known in the art. Typically it is intended to maintain the highest purity for the water stream by its re-passage through one or more of the purification processes or technologies, and by its continual movement, thereby preventing stagnation and the opportunity for any remaining bacteria and/or micro organisms to adhere to a surface and grow.

Recirculating the purified water stream, and/or any water supply stream that has entered the water purification apparatus prior to the stopping of the dispense of the purified water stream and which is downstream of the point of recirculation, provides a recirculating water stream. This is able to pass through the major parts or stages or portions of the water purification apparatus, generally including at least the same pump, the oxidiser and optionally one or more de-ionisers.

The first part of the recirculating water stream, comprising the purified water stream created but not dispensed, then re-passes through the same oxidiser and provides a re-oxidised water stream. The re-oxidised water stream may continue to be recirculated, and/or be provided to the point of dispense in a manner known in the art.

Step (b) of the present invention comprises measuring a conductivity value, as defined hereinabove, of the water stream prior to the oxidiser to be used to determine the TOC of the purified water stream.

Apparatus and devices for measuring a conductivity value of a water stream as a first or other conductivity sensor are known in the art, and include in-line conductivity cells. One or more such apparatus or devices may already be a part or component of the water purification apparatus, such that they could be used for measuring the conductivity value of the re-oxidised stream, as well as conductivity values of the supply, intermediate and purified water streams.

The relationship between TOC and the conductivity change generated is a function of the oxidising device's properties, its housing's geometry, the rate of flow and the concentration of the species in the water stream entering the oxidiser. The change in conductivity will also be a function of the conductivity of the water stream entering the oxidiser. These effects can be determined experimentally for the actual components being used, and a calibration can be produced to provide a known or expected level of oxidation of organic substances during standard and/or normal operation of the oxidiser. Preferably a comparison is made with the conductivity of ultra-purified water as the subsequent change in conductivity is then greatest.

Hitherto, the efficiency of an oxidiser could be estimated by periodically increasing the time the recirculated water stream spent in the oxidiser to try and achieve complete oxidation of the or any organic substances present. Then the expected relationship between a change in conductivity during normal operation and the change in conductivity during complete oxidation could be used to check the efficiency of the oxidation and the values being used in the algorithms, and modify these values or alert the user such as raising an alarm, as necessary.

Thus, the TOC in the water stream prior to the oxidiser could be estimated by measuring the conductivity value of the stream before and after the oxidiser, usually by a simple line cell, and/or assuming a conductivity value based on known or expected provision of the purified water stream and/or operation of the water purification apparatus, such as 0.055 μS/cm. By carrying out this process on recirculating water, the TOC of the water at the outlet of the unit can be determined. But this requires knowledge of the efficiency of the oxidiser.

The present invention seeks to improve the conventional practice.

Step (c) of the method of the present invention can be carried out by any suitable control or operation, typically a signal from a suitable controlling apparatus or program to turn off and stop the first pump.

Step (d) of the method of the present invention uses a second pump. The second pump may be the same or similar to the first pump, although typically having a smaller operating power as it is only required to circulate or re-circulate water around a smaller circuit than the first pump. The first pump is intended to provide the drive or pumping through the remainder of the water purification apparatus.

The second pump is dedicated to operating only for water in and around the oxidiser recirculation circuit. The second pump is not able or intended to allow the method or apparatus of the present invention to provide a purified water stream to a point of dispense as per step (a). Furthermore, the second pump is not able or intended to allow the method or apparatus of the present invention to recirculate a purified water stream around a main recirculation circuit as discussed herein.

The oxidiser re-circulation circuit is formed to re-circulate water that has passed through the oxidiser, back to a point or stage of the water purification apparatus that is only upstream of the first conductivity sensor. This is to re-circulate only the water that is present in the oxidiser re-circulation circuit, in the oxidiser, and in those portions of the water purification apparatus that encompasses the inlet and outlet of the first conductivity sensor and the inlet and the outlet of the oxidiser. This oxidises the water in the oxidiser re-circulation circuit to a higher degree, and so generates a greater conductivity difference than would occur with a single pass. Optionally, the oxidiser re-circulation circuit requires filling if not already filled by the first pump. Optionally, the method involves one or more further valves, able to assist the introduction of further water if required into the oxidiser re-circulation circuit during step (d).

The water that is in the oxidiser re-circulation circuit may be directed into it from the inlet of the water purification apparatus or from the point of dispense or from a cartridge containing water to be tested or a system suitability solution as described elsewhere herein.

Step (d) of the method of the present invention involves circulating or re-circulating the water within the oxidiser re-circulation circuit only through the first conductivity sensor and the oxidiser, a plurality of times. Thus, the only purification action being applied to the water is provided by the oxidiser, as the oxidiser re-circulation circuit is dedicated to providing only a pathway through the oxidiser and the first conductivity sensor.

The water in the oxidiser recirculation circuit is intended or expected to be increasingly oxidised by its multiple passages through the oxidiser.

Optionally, the oxidiser re-circulation circuit includes a non-return valve, able to prevent circulation of water through the oxidiser re-circulation circuit (not being part of the main water purification apparatus pathway) when not required.

Step (e) of the method of the present invention is carried out using the same first conductivity sensor to measure a second conductivity value of the re-circulated water in the oxidiser re-circulation circuit. Such water has now passed through the oxidiser a plurality of times, such that the second conductivity value now measured by the first conductivity sensor will typically be different to the first conductivity sensor (unless the oxidiser is not carrying out any further oxidising, and is therefore shown to be inefficient, possibly redundant, and needing replacement).

For step (f), a calculation involving the first conductivity value and the second conductivity value provides a change in the TOC of the water circulated in the oxidiser re-circulation circuit, which can then be used to calculate the conductivity of the water prior to step (d), and thereby the efficiency or effectiveness of the oxidiser.

Thus, the present invention provides confirmation to a user and/or service personnel of the performance of the oxidiser, and possibly the need to service and/or replace the oxidiser based on its expected performance.

The present invention also allows the method and apparatus, either automatically or at the instigation of a user and/or service personnel, to switch the determination of the TOC of a purified water stream in a water purification apparatus from a relatively normal level, such as up to 50 ppbC or 100 ppbC, to now detect or determine the TOC of a water stream being above such levels with greater accuracy. This switch can be used to check the calibration of the oxidiser by operation and comparison of TOC determinations with and without use of the oxidiser recirculation circuit, and by comparison of the measured change in conductivity and subsequent derived TOC values.

The present invention also allows the method and apparatus to be used for the determination of the TOC of a different or separate water source or stream, that could be added into the water purification apparatus at an appropriate location, such as but not limited to, prior to the oxidiser. Where the water purification apparatus has a deioniser prior to the oxidiser, such separate water could be added to a replacement to the deioniser, using the same ports, for supply of such water into the oxidiser prior to step (d).

Optionally, step (a) of the method of the present invention further comprises using the first pump to pass the water supply stream through one or more de-ionisers located after the oxidiser, to provide a further purified water stream, either for dispense as a dispense purified stream, or passage around a main recirculation circuit for recirculation upstream of the first conductivity sensor.

The main re-circulation circuit is typically intended to maintain in a manner known in the art a predetermined or desired water purity for water that has passed through the stages or processes of the water purification apparatus and is ready for dispense, but for which no dispense is currently required. Thus, the main re-circulation circuit can circulate or re-circulate such purified water, in order to maintain its purity at the or a predetermined or desired level, ready for dispense when required. Typically, the first pump is able to provide the drive required to also pass the purified water around the main re-circulation circuit. The main re-circulation circuit typically extends from a suitable valve at the point of dispense of the purified water, which generally operates to either dispense or re-circulate the purified water, to a part of the water purification apparatus downstream the initial provision of a water supply stream and upstream of the first pump.

In order to maximise changes in conductivity it is preferable for the conductivity of the first conductivity value to be low, preferably <1 μS/cm, <0.1 μS/cm or ultrapure water of 0.055 μS/cm.

Optionally, step (a) further comprises passing the water supply stream through a deioniser prior to the first conductivity sensor and the oxidiser. Deionisers are discussed herein. The deioniser may assist purifying a water supply stream prior to the present invention, especially if there has been little or insufficient pre-purification of the water supply stream, i.e. the quality of the water supply stream is considered to be too low before the ‘polishing’ provided by the water purification apparatus of the present invention.

As discussed herein, the present invention can provide a method of calculating a ‘usual’ or expected ‘low level’ TOC expected for most water supply streams, such as up to 50 ppbC, and switching to measuring a relatively high or higher TOC, such as from 50 ppbC up to 500 ppbC. Thus, alternatively or additionally, the deioniser may be used when the present invention is intended to calculate a TOC above a pre-determined value such as 50 ppbC, in particular up to a higher pre-determined value, such as 500 ppbC.

Optionally, the present invention comprises a bypass stream or pathway, and a deioniser stream or pathway, between the first pump and the first conductivity sensor. Thus, the present invention can determine via suitable valves located in each stream or pathway the passage of water from the first pump to the first conductivity sensor.

Optionally, the present invention further comprises the step of measuring a conductivity value of the post-oxidiser water stream to determine the TOC of the purified water stream prior to dispense.

Measuring the conductivity value of the post-oxidiser water stream assists in relation to the method of determining the TOC of a purified water stream as shown and described in WO2010/043896 A, incorporated herein by way of reference, so that the TOC of the purified water stream can be provided without requiring a dedicated TOC monitor at the point of dispense of the purified water stream.

Optionally, the method of the present invention is able to calculate the TOC of above a pre-determined value, such as 100 ppbC, 200 ppbC, 300 ppbC, 400 ppbC or 500 ppbC, for the water from the oxidiser. Thus, in one embodiment of the present invention, the method could be used to determine if the TOC post-oxidiser is above a certain predetermined level, such as 100 ppbC, and require operation of the method of the present invention in order to determine if the TOC value calculated is because of the deterioration in the performance of the oxidiser. For example, if the TOC calculated by step (f) of the method of the present invention indicates a TOC result of ≥100 ppbC, the present invention could seek to repeat the method of the present invention until either the TOC value is lower than 100 ppbC, or require the servicing and/or replacement of the oxidiser, followed by optional repeat of the method of the present invention to confirm that such action has resulted in a lowering in the TOC below a predetermined level.

Thus, the present invention is able to be calibrated to measure a water supply stream having a TOC up to 500 ppbC. This allows the method of the present invention to provide a “system suitability test” such as described in USP643 of the United States Pharmacopeial Convention, (and described on page 522 of the USP40 version of USP643 issued on Dec. 1, 2017), and to confirm whether the water purification apparatus is able to accurately quantify levels of TOC up to 500 ppbC. Calibration and operation of the present invention could be extended beyond 500 ppbC, such as up to 1000 ppbC.

Optionally, the present invention provides a method of calculating the usual or expected TOC relatively ‘low level’ type of analysis of up to a common or typical value expected for most water supply streams, such as up to 50 ppbC, and switching to a relatively high or higher TOC level analysis, such as from 50 ppbC up to 500 ppbC. Thus, normal or expected use of the water purification apparatus typically involves only requiring a low TOC level analysis.

Optionally, the present invention further comprises a method as claimed in any one of the preceding claims further comprising the step of inserting a TOC-standard cartridge into the water purification apparatus upstream of the first conductivity sensor prior to step (d), to provide a TOC-standard concentration prior to steps (e) and (f).

TOC standard cartridges are known in the art. One cartridge can contain a 500 ppbC solution of sucrose. Another known TOC-standard cartridge contains 500 ppbC solution of benzoquinone. Such cartridges can be installed in order to provide a predetermined or known level of TOC, which can be used in step (d) part of the method of the present invention, to provide a measurement of the second conductivity value that should be expected for the water circulating in the oxidiser re-circulation circuit. Calibration of the oxidiser can then be confirmed or adjusted, prior to return to normal use of the water purification apparatus.

According to another aspect of the present invention, there is provided a water purification apparatus comprising:
a first pump,
a first conductivity sensor,
an oxidiser, and
an oxidiser recirculation circuit having a dedicated second pump,
   wherein the first pump is able to pass a water supply stream through the water purification apparatus, including through the first conductivity sensor and the oxidiser, to provide a purified water stream available for dispense;
the first conductivity sensor is able to measure a first conductivity value of the water supply stream prior to the oxidiser;
the second pump is only able to recirculate water through the oxidiser recirculation circuit, the first conductivity sensor and the oxidiser a plurality of times, to allow the first conductivity sensor to measure a second conductivity value of the recirculated water prior to the oxidiser; and
the first conductivity value is able to be compared with the second conductivity value to calculate a TOC of the water in the oxidiser prior to operation of the second pump.

The water purification apparatus can be used to provide a method of determining the total organic carbon content (TOC) as described herein.

Optionally, the water purification apparatus further comprises a one or more de-ionisers and a main recirculation circuit as described herein.

Optionally, the water purification apparatus further comprises a second conductivity sensor located between the oxidiser and the one or more de-ionisers as described herein.

Optionally, the water purification apparatus is able to measure a TOC of up to 500 ppbC as described herein.

Optionally, in the water purification apparatus, the oxidiser comprises one or more ultraviolet light emitters as described herein.

Optionally, in the water purification apparatus, the capacity of the oxidiser recirculation circuit comprises >20% of the capacity of the main recirculation circuit, optionally >5% of the capacity of the main recirculation circuit.

Optionally, the water purification apparatus further comprises a location for the introduction of a TOC-standard cartridge upstream of the first conductivity sensor.

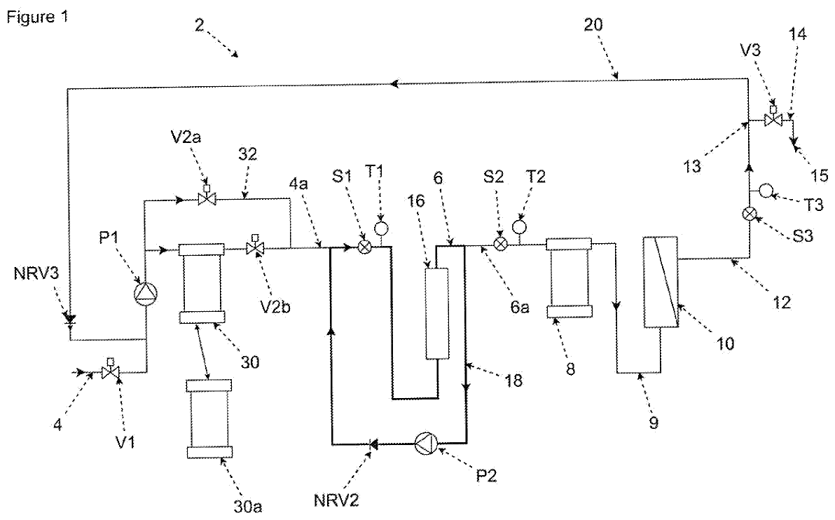

An embodiment of the present invention will now be described by way of example only, and with reference to the accompanying drawing, FIG. 1, which schematically shows a method of determining the TOC of a purified water stream according to one embodiment of the present invention.

Referring to FIG. 1, FIG. 1 shows a method of determining the TOC of a purified water stream 6 in a water purification apparatus 2 as described hereinafter.

The water purification apparatus 2 may have any number of further purification means, units or devices not shown in FIG. 1. The water purification apparatus 2 starts with a water supply stream 4 that can be provided from a source such as the potable mains. The water supply stream 4 may have been treated by one or more steps and/or processes (not shown) to help reduce impurities in a manner known in the art, such as one or more pre-treatment filters or membranes and de-ionisers, and by means such as reverse osmosis, capacitive deionisation or ion exchange such that the conductivity of the water supply stream is <30 μS/cm, to help reduce impurities. Preferably, the conductivity of the water supply stream is <10 μS/cm, or <1 μS/cm, or even <0.1 μS/cm.

The water supply stream 4 passes through a first valve V1, a first pump P1, and then either through a deioniser 30 containing ion exchange resin and control valve V2b, or through a bypass line 32 and a second valve V2a, to arrive as a water supply stream 4a upstream of the first conductivity sensor S1 and a first temperature sensor (T1), in a manner known in the art.

The deioniser 30 provides a suitable additional purification of the water either from the water supply stream 4, which such stream 4 is of insufficient quality as discussed, or it is desired to measure the TOC of the water in the main recirculation circuit 20, (discussed further hereinafter).

The bypass line 32 provides a pathway where purification by a deioniser is not desired, in particular where it is intended for the present invention to measure TOC at a relatively high TOC level, such as between 50-500 ppbC.

The location of the deioniser 30 also provides a suitable location for the temporary replacement of a deioniser cartridge by a TOC-standard cartridge as discussed hereinafter.

Valves 2a and 2b determine the pathway for water to a position 4a before the oxidiser re-circulation circuit 18 as discussed hereinafter, and the valves V2a and V2b can therefore assist the switching of the water purification apparatus 2 between its different types of TOC measurements.

The first conductivity sensor S1 measures a first conductivity value of the water supply stream 4a, following which the water supply stream 4a passes into an oxidiser 16. The oxidiser 16 comprises one or more ultraviolet light emitters, which oxidise organic compounds or substances in the water supply stream 4a to create ionised species in a manner known in the art. The post-oxidiser water stream 6 passes through a second conductivity sensor S2 and temperature sensor T2, where a further or third conductivity value can be measured. This is discussed in more detail below.

In normal operation, the post-oxidiser stream 6 then passes into one or more de-ionisers to remove the ions or ionisable species generated in the oxidiser, such as a de-ionisation unit 8, comprising one or more ion exchangers, to provide a further purified water stream 9. Such stream 9 can then pass through an ultra-filtration membrane 10 for polishing, to provide a final purified water stream 12. Stream 12 can have a conductivity value measured by a third conductivity sensor S3 and temperature sensor T3, prior to reaching a split point 13. If desired, the purified water is available for dispense by partially or fully opening of valve V3 to provide a purified dispense stream 14 available from a point of use outlet 15.

When dispense is not required, valve V3 can be shut, to allow the purified water stream 12 to be re-circulated in a main re-circulation circuit 20, which re-circulates the stream 12 back to a point in the pathway of the water purification apparatus 2 downstream of the valve V1 and upstream of the first pump P1, for return through the pump P1 and valve V2 in a manner described hereinbefore. Thus, when purified water 12 is not required, the re-circulated water maintains the water quality of water in the water purification apparatus 2 at a level that is ready for immediate dispense at any time. By partial operation of valve V3 a desired flow of water may be passed to the point of use 15 with the rest recirculated in the main re-circulation circuit 20.

The units and operations described above are all well known in the art. The use of the first and third conductivity values measured by the first and second conductivity sensors S1 and S2 provide a ‘low level’ TOC analysis in the water purification apparatus, and more particular across the oxidiser 16, in order to achieve monitoring of the efficiency of the oxidiser 16.

In a first use of the method of the present invention, the first pump P1 and the valve V2 are in-activated by a controller or control means. Then, the second pump P2 is activated, such that the water in the oxidiser re-circulation circuit 18 (emphasised for clarity by the bolder lines in FIG. 1), is now re-circulated back to a position in the water stream pathway upstream of the first conductivity sensor S1. In this way, the oxidiser re-circulation circuit 18 is only re-circulating water from the oxidiser 16 through the first conductivity sensor S1 and the oxidiser 16. The non-operation of the pump P1 prevents such water flowing towards the second conductivity sensor S2.

By re-circulating the water around the oxidiser re-circulation circuit 18 using pump P2 a number of times, such as two, three, four, five, six, seven, eight, nine, ten, or more than ten times, the water is repeatedly subjected to oxidation and by having an increased flowrate, such water undergoes increasing turbulence through the oxidiser 16, to improve the oxidation efficiency, and to oxidise a further percentage of any organic compounds or substances within such water, relative to the water supply stream 4a upstream of the first conductivity sensor S1.

After a period of time, such as a number of seconds or minutes, the second pump P2 is inactivated, and the first conductivity sensor S1 measures a second conductivity value of the re-circulated water in the oxidiser re-circulation circuit 18.

A suitable program can then calculate the TOC from the change in the conductivity of the re-circulated water relative to the conductivity measured from the first conductivity sensor prior to using the oxidiser re-circulation circuit 18.

In the alternative, if the TOC has a value of <100 ppbC, the water purification apparatus 2 can continue in a manner known in the art by reactivation of the first pump P1 and opening of the valve V2. A non-return valve NRV2 in the oxidiser re-circulation circuit 18 prevents flow occurring through the oxidiser re-circulation circuit during normal operation of the water purification apparatus 2 based on the first pump P1.

Values for TOC obtained with and without the use of the oxidiser re-circulation circuit 18 can be compared as a means of re-calibrating the algorithms used by the controller.

The oxidiser re-circulation circuit 18 may be used to determine the TOC of the water supply stream 4 by it being filled via line 32 and 4a.

The oxidiser re-circulation circuit 18 may be used to determine the TOC of the water as dispensed from the point of use outlet 15, being the same water quality as that at the split point 13, by it being filled from the split point 13 via line 20, 32 and 4a.

In a second use of the method of the present invention, a TOC system suitability test can be carried out. Step (a) and (b) of the method of the present invention are described above, and step (c) can be carried out in the same way in order to stop the first pump P1 and deactivate valve V2.

Using the same method steps as described above, provides the user or service engineer with a TOC value which can be seen as a ‘blank’ value.

With the second pump P2 deactivated, a user or service engineer can then insert a TOC-standard cartridge 30a in place of the deionisation pack 30, (or into separate suitable inlet and outlet ports within the pathway of the water purification apparatus 2 upstream of the first conductivity sensor S1).

The first pump P1 is then activated for a short time, in order to load the standard solution in the TOC-standard cartridge 30a into the oxidiser re-circulation circuit 18. First pump P1 is then in-activated, and the second pump P2 is activated for a certain period of time, especially to circulate the water in the oxidiser re-circulation circuit 18 a number of times through the oxidiser 16. The second pump P2 is then deactivated or stopped, and a measurement is taken by the first conductivity sensor S1 to provide a conductivity value related to the substance in the TOC-standard cartridge 30a.

Data tags on the cartridges may be used to inform the water purification apparatus controller that a TOC standard cartridge 30a has been fitted and to proceed as required.

TOC-standard solutions are specified as sucrose and benzoquinone in USP 643, but other specified chemicals can be used as necessary. Standard concentration of these substances can be provided, such as a 500 ppbC solution as specified in USP 643. As these are well known substances undergoing a standard process, they should be oxidised in an expected degree by an oxidiser working at an expected efficiency, to provide an expected conductivity value after circulation a number of times through the oxidiser 16 and variation from this can be used to enable re-calibration or as an indication of an error in the system.

One, two, or more TOC-standard cartridges can be used, with known flushing and cleaning of the oxidiser thereinbetween, in order to provide a number of conductivity values that can be gauged against expected conductivity values in relation to substance. These can be such as sucrose and benzoquinone at an initial TOC of 500 ppbC.

Further cleaning, flushing and possible installation of other purification apparatus or packs then allow the water purification apparatus to be returned or started to provide a purified dispense stream in a manner known in the art.

### Example 1

Example 1 illustrates TOC monitoring using the apparatus detailed in FIG. 1 via the methods described herein.

In general use, the valve V2a is open and valve V2b is closed to allow a bypass of the ion-exchanger 30. Pump P1 thus passes water at 1 l/min from either the dispense point 13 or from inlet point 4 through Valve V2a, as desired, and into first conductivity sensor S1 and temperature sensor T1. The conductivity is measured at the first conductivity sensor S1 to provide a first measurement.

As part of the present invention, Pump P1 is then switched off, and the second pump P2 and oxidiser 16 are switched on for a set period of time to circulate water only through the recirculation circuit 18, including the oxidiser 16, a number of times.

The second pump P2 and the oxidiser 16 are then switched off for a further set period of time before taking a second conductivity measurement is taken from the first conductivity sensor S1.

The increase in conductivity between the first and second measurements is calculated and compared to a ‘calibration look-up’ table to derive the TOC in the water that was directed into the oxidiser from either dispense point 13 or from inlet point 4

### Example 2

Example 2 illustrates determination of TOC of a water sample contained within a container using the apparatus detailed in FIG. 1 via the methods described herein.

A replacement container is filled with a separate water sample to be tested. The deioniser 30 is replaced with the container. The valve V2a is closed and valve V2b opened. Pump P1 delivers water at 1 l/min from the dispense point 13 or from inlet point 4 through Valve V2b and into sample container 30a. The contents of the container are thus moved from the container 30a to fill first conductivity sensor S1, temperature sensor T1 and oxidiser 16. The conductivity of the water sample is measured at the first conductivity sensor S1 to provide a first measurement.

As per Example 1, the Pump P1 is switched off, and the second pump P2 and oxidiser 16 are switched on for a set period of time to circulate the water sample only through the recirculation circuit 18, including the oxidiser 16, a number of times.

The second pump P2 and the oxidiser 16 are then switched off for a further set period of time before taking a second conductivity measurement is taken from the first conductivity sensor S1.

The increase in conductivity between the first and second measurements is calculated and compared to a ‘calibration look-up’ table to conclude TOC of the water sample.

### Example 3

To conduct a system suitability test as described herein, such as to test the capability of apparatus against the requirements of USP643, the deioniser 30 is replaced by a TOC-standard cartridge 30a, in order to supply a solution stipulated in the system suitability test.

Operation of the apparatus is then the same as described in relation to Example 2 above. Pump P1 delivers water at 1 l/min from the dispense point 13 or from inlet point 4 through Valve V2b and into TOC-standard cartridge 30a. The contents of the container are thus moved from the container 30a to fill first conductivity sensor S1, temperature sensor T1 and oxidiser 16. The conductivity of the water sample is measured at the first conductivity sensor S1 to provide a first measurement.

As per Example 2, the Pump P1 is switched off, and the second pump P2 and oxidiser 16 are switched on for a set period of time to circulate the water sample only through the recirculation circuit 18, including the oxidiser 16, a number of times.

The second pump P2 and the oxidiser 16 are then switched off for a further set period of time before taking a second conductivity measurement is taken from the first conductivity sensor S1.

The increase in conductivity between the first and second measurements is calculated and compared to the calibration requirements of USP643 to determine if the apparatus is able to “periodically demonstrate” that it can provide a suitable response when fed with a level of 500 ppbC of specified organic molecules (as defined therein).

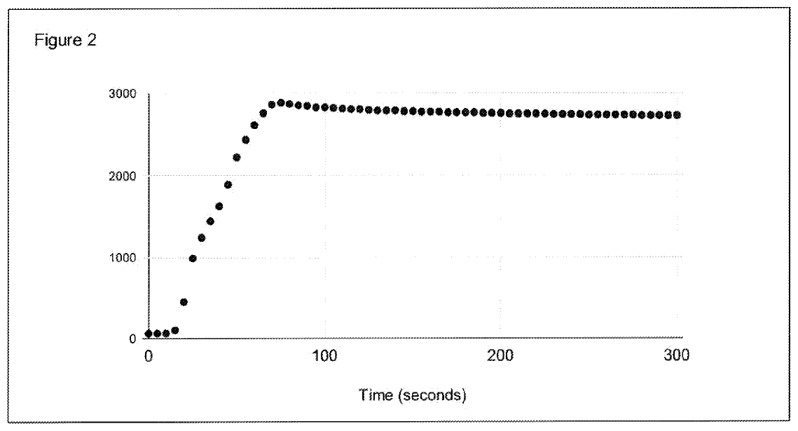

FIG. 2 shows a graph of conductivity measurements by the first conductivity sensor S1 in FIG. 1 over time. At the beginning of the graph (time 0-15 seconds), the conductivity is very low; This value is recorded as the 1st conductivity measurement and is of the water delivered by pump P1 directly to sensor S1 and into oxidiser 16; during this initial time period pump P2 is not operated to re-circulate water in oxidiser re-circulation circuit 18. At 20-seconds, P1 is switched off and P2 switched on, water is now passed repeatedly through the oxidiser 16, oxidiser recirculation circuit 18 and sensor S1.

As time progresses until 80 seconds, it can be seen that there is an increasing conductivity value measured at sensor S1. At 80 seconds pump P2 and Oxidiser 16 are switched off. Sensor S1 continues to measure conductivity until stabilised, and a second conductivity value is recorded at 300 seconds. The difference between the first and second recorded conductivity values is calculated. From this graph, the skilled man can then calculate the concentration of TOC contained in the water delivered by pump P1 into oxidiser 16 by comparing the difference between the first and second recorded conductivity values and predetermined, calibrated values, specific to the particular apparatus. In the particular example shown in FIG. 2, the water delivered by pump P1 into the oxidiser contained TOC at a concentration of 500 ppbC.

The present invention provides a method, apparatus and system that can directly determine the effectiveness of the oxidiser to reduce the TOC in the water stream prior to dispense. This provides the user or service personnel with immediate and reliable determination of the status or efficiency of the oxidiser.

The present invention provides a method, apparatus and system that can achieve a high level TOC analysis at any time required, to at least match the requirements of USP643.

What is claimed is:

1. A method of determining the total organic carbon content (TOC) of a purified water stream in a water purification apparatus having at least a first pump, a first conductivity sensor, an oxidiser, and an oxidiser recirculation circuit having a dedicated second pump, the method comprising at least the steps of:
(a) using the first pump (P1) to pass a water supply stream through the water purification apparatus, including through the first conductivity sensor and the oxidiser, to provide a purified water stream available for dispense;
(b) using the first conductivity sensor to measure a first conductivity value of the water supply stream prior to the oxidiser;
(c) stopping the first pump;
(d) using the second pump in the oxidiser recirculation circuit to recirculate the water in the oxidiser only through the first conductivity sensor and the oxidiser a plurality of times,
(e) using the first conductivity sensor to measure a second conductivity value of the recirculated water; and
(f) calculating the TOC of the water in the oxidiser, prior to step, from the first and second conductivity values.

2. A method as claimed in claim 1 further comprising providing the water in the oxidiser recirculation circuit for step from the water supply stream that entered the water purification apparatus in step (a).

3. A method as claimed in claim 1 further comprising providing the water in the oxidiser recirculation circuit for step (d) from the purified water stream available for dispense.

4. A method as claimed in claim 1 further comprising the step of inserting a water-filled cartridge into the water purification apparatus upstream of the first conductivity sensor prior to step, and using said water in step (d).

5. A method as claimed in claim 1 wherein step (a) further comprises using the first pump to pass the water supply stream through one or more de-ionisers located after the oxidiser, to provide a further purified water stream, either for dispense as the purified water stream, or for passage around a main recirculation circuit for recirculation upstream of the first pump.

6. A method as claimed in claim 1 wherein step (a) further comprises passing the water supply stream through a deioniser prior to the first conductivity sensor and the oxidiser.

7. A method as claimed in claim 1 further comprising the step of measuring a conductivity value of the post-oxidiser water stream to determine the TOC of the purified water stream prior to dispense.

8. A method as claimed in claim 1 able to calculate a TOC of above 100 ppbC for the water prior to the oxidiser.

9. A method as claimed in claim 8 able to calculate a TOC of up to 500 ppbC for the water prior to the oxidiser.

10. A method as claimed in claim 1 wherein the oxidiser provides UV irradiation.

11. A method as claimed in claim 1 further comprising the step of inserting a TOC-standard cartridge into the water purification apparatus (2) upstream of the first conductivity sensor (S1) prior to step (d), to provide a TOC-standard concentration prior to steps (e) and (f).

12. A method as claimed in claim 11 further comprising providing the water in the oxidiser recirculation circuit (18) for step (d) from the TOC-standard cartridge.

13. A method as claimed in claim 11 further comprising using the first conductivity sensor (S1) to measure a third conductivity value of the recirculated water after inserting the TOC-standard cartridge, and comparing the first conductivity value to the third conductivity value to provide a standard-comparison TOC.

14. A method as claimed in claim 1 further comprising repeating steps (a) to (f) if the TOC is ≥100 ppbC.

15. A water purification apparatus comprising:
a first pump,
a first conductivity sensor,
an oxidiser, and
an oxidiser recirculation circuit having a dedicated a second pump,
wherein the first pump is able to pass a water supply stream through the water purification apparatus, including through the first conductivity sensor and the oxidiser, to provide a purified water dispense stream available for dispense;
the first conductivity sensor is able to measure a first conductivity value of the water supply stream prior to the oxidiser;
the second pump is only able to recirculate water through the oxidiser recirculation circuit, the first conductivity sensor and the oxidiser a plurality of times, to allow the first conductivity sensor to measure a second conductivity value of the recirculated water prior to the oxidiser; and
the first conductivity value is able to be compared with the second conductivity value to calculate a TOC of the water in the oxidiser prior to operation of the second pump.

16. A water purification apparatus as claimed in claim 15 further comprising a one or more de-ionisers and a main recirculation circuit.

17. A water purification apparatus as claimed in claim 16 further comprising a second conductivity sensor located between the oxidiser and the one or more de-ionisers.

18. A water purification apparatus as claimed in claim 15 able to measure a TOC of up to 500 ppbC.

19. A water purification apparatus as claimed in claim 15 wherein the oxidiser comprises one or more ultraviolet light emitters.

20. A water purification apparatus as claimed in claim 15 wherein the capacity of the oxidiser recirculation circuit (18) comprises >20% of the capacity of the main recirculation circuit, optionally >5% of the capacity of the main recirculation circuit (20).

21. A water purification apparatus as claimed in claim 15 further comprising a location for the introduction of a TOC-standard upstream of the first conductivity sensor (S1).

22. A water purification system comprising an initial water purification apparatus able to provide a water supply stream having a conductivity <30 μS/cm, and a water purification apparatus as defined in claim 15.
